(19) United States
(12) Patent Application Publication
(10) Pub. No.: US 2003/0125782 A1
(43) Pub. Date: Jul. 3, 2003

(54) Methods for the regeneration of bone and cartilage

(76) Inventor: Jackson Streeter, Reno, NV (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/299,042

(22) Filed: Nov. 15, 2002

Related U.S. Application Data
(60) Provisional application No. 60/335,727, filed on Nov. 15, 2001.
(60) Provisional application No. 60/341,464, filed on Dec. 17, 2001.
(60) Provisional application No. 60/344,932, filed on Dec. 21, 2001.
(60) Provisional application No. 60/354,007, filed on Jan. 31, 2002.

(51) Int. Cl. A61N 1/05

(52) U.S. Cl. 607/88; 604/20; 607/50

(57) ABSTRACT

Therapeutic methods for regenerating bone and cartilage are described, the methods including delivering a tissue regenerative effective amount of light energy having a wavelength in the visible to near-infrared wavelength range to a site of injured or damaged bone or cartilage. The tissue regenerative effective amount of light energy is a predetermined power density (mW/cm²) received at the site, and is determined by selecting a dosage and power of the light energy sufficient to deliver the predetermined power density of light energy to the site of damage or injury. The light to methods are further applicable to in vitro or in vivo growth of cartilage replacement tissue on a biocompatible three-dimensional scaffold.

### RELATED APPLICATION INFORMATION

This application claims priority under 35 U.S.C. §119(e) to U.S. Provisional Application Serial No. 60/335,727, filed Nov. 15, 2001, U.S. Provisional Application Serial No. 60/341,464, filed Dec. 17, 2001, U.S. Provisional Application Serial No. 60/344,932, filed Dec. 21, 2001, and U.S. Provisional Application Serial No. 60/354,007, filed Jan. 31, 2002, and also claims priority under 35 U.S.C. §120 to U.S. patent application Ser. No. 10/287,432, filed Nov. 1, 2002, the disclosures of which are hereby incorporated by reference in their entireties.

### BACKGROUND OF THE INVENTION

The present invention relates in general to the medical procedures for treating injured or damaged bone and cartilage, and more particularly to methods for regenerating bone and cartilage using light therapy.

Osteogenesis (bone regeneration) and chondrogenesis (cartilage regeneration) are highly complex biological processes with significant relevance to the treatment of injuries and disease. Typically, fractured bones are simply set, immobilized, and monitored over a period of several weeks or even months as the normal process of bone remodeling heals the fracture. Graft procedures are also used to treat injured or damaged bone when the normal remodeling process is for some reason insufficient. In developed nations more than a million bone grafting procedures are performed annually to repair bone that is damaged due to trauma or degenerative bone disease. In addition, bone grafting has recently seen widespread application in the field of dentistry due to the popularity of dental implants.

Conventional bone tissue regeneration is achieved by filling a bone defect site (recipient graft site) with either autologous or allograft material and covering the graft material with a barrier material to exclude competitive cells. The barrier material, typically a poly-tetrafluoroethylene (PTFE) membrane, functions as a physical barrier to protect the graft material from disruption over time, to retard the ingrowth of unwanted tissue into the graft material and to allow cells to migrate into the recipient graft site from adjacent osseous tissues. However, barrier materials like PTFE are not biodegradable, must be removed in an additional surgery, and also increase the risk of infection if complete soft tissue coverage is not obtained.

The type of graft material used determines how bone tissue is regenerated. Conventional graft material includes live active bone tissue, such as fresh autogenous cancellous bone and marrow, which includes osteoblasts that form new bone. Live active bone tissue also induces undifferentiated cells in the recipient graft site to differentiate into osteoblasts that form additional new bone. Demineralized, freeze-dried, allogenic bone (“DFDBA”), an inducing graft material, has also been used as a graft material. DFDBA induces undifferentiated cells in the graft site to differentiate into osteoblasts and grow into new bone, while the graft material itself is resorbed by the host. Autogenous cortical bone chips have also been used, as a type of scaffolding graft material to passively attract osteoblasts native to the recipient graft site where the cells may grow into new bone.

However, great variation exists in the success of bone grafting procedures due to a variety of factors including the condition of the graft site, graft material and processing, and immunological compatibility between donor and recipient in allograft procedures. Autologous live tissue bone grafts generally produce excellent results and avoid compatibility problems, but do require additional surgery on the patient to harvest the graft material. Particularly when the donor is also the recipient, such live tissue may be in limited supply. In addition, the additional surgery carries the risks of significant post-operative pain, hemorrhage and infection. Moreover, the other conventional graft materials do not always provide reliable bone tissue regeneration because they are not capable of inducing sufficient recipient graft site bone formation before competitive soft tissue and epithelial cells fill the recipient graft site.

Cartilage regeneration and replacement procedures are even more problematic. Unlike the osteogenesis that normally occurs to repair damaged. bone, chondrogenesis does not normally occur to repair damaged cartilage tissue. However, methods for making or repairing cartilage have been described, including the use of a biocompatible three-dimensional scaffold for growing cartilage in vitro or for implanting into a patient at a site of cartilage damage or loss in a patient and growing the cartilage in vivo. For example, periosteal or perichondrial tissue is attached to the scaffold to hold the scaffold in place and to provide a source of chondrocyte progenitor cells, chondrocytes and other stromal cells. A preparation of cells that can include chondrocytes, chondrocyte progenitor cells or other stromal cells is administered, either before, during or after implantation of the scaffold and/or the periosteal/perichondrial tissue; and the cells are also administered directly into the site of the implant in vivo to promote chondrogenesis is and the migration of chondrocytes, progenitor cells and other stromal cells from the surrounding tissue into the scaffold for to form new cartilage at the site of implantation. However, chondrogenesis does not always occur to meaningful levels. Frequently, the most effective treatment for damaged cartilage is full prosthetic joint replacement using artificial implants.

These and other difficulties with presently available bone-grafting, bone regeneration and cartilage regeneration procedures have prompted investigators to pursue study of the cellular and molecular bases of osteogenesis and chondrogenesis. In the typical course of differentiation a pluripotent stem cell proceeds through one or more intermediate stage cellular divisions, finally resulting in the appearance of one or more specialized cell types. Stem cell lineages present in marrow include hematopoietic, mesenchymal, and stromal. The uncommitted cell types that precede the fully differentiated forms, and which may or may not be true stem cells, are defined as precursor cells. Recent research has identified and isolated the more primitive, less specialized, bone and cartilage precursor cells from marrow and other tissues from which arise specialized, committed cell types.

The precise nature and extent of signals that trigger differentiation down a particular path are not fully understood, but it is clear that a variety of chemotactic cellular, and other environmental signals are involved. Within the mesenchymal lineage, for example, mesenchymal stem cells (MSC) cultured in vitro can be induced to differentiate into bone or cartilage in vivo and in vitro, depending upon the tissue environment or the culture medium into which the cells are placed. MSC thus have the capacity to differentiate into a variety of different cell types including cartilage, bone, tendon, ligament, and other connective tissue types. Hematopoietic stems cells (HSC) have the capacity for self-regeneration and for generating all blood cell lineages while stromal stem cells (SSC) have the capacity for self-renewal and for producing the hematopoietic microenvironment.

Researchers have most commonly used periosteum and marrow as sources of precursor cells having osteogenic potential. Most researchers use cells isolated from periosteum for in vitro assays. Similarly, the most common sources of cartilage precursor cells to date have been periosteum, perichondrium, and marrow. Cells isolated from marrow have also been used to produce cartilage in vivo. Periosteal and perichondral grafts have also been used as sources of cartilage precursor cells for cartilage repair. Methods are known that involve in vitro culturing to isolate and amplify mesenchymal stem cells (MSC) from marrow. The resulting in vitro amplified, marrow-isolated MSC can then be introduced into a recipient at a transplantation repair site.

However, even the most current methods for preparing graft material from precursor cells using in vitro culture techniques remain limited. First, all methods still require that some bone marrow or other tissue be harvested, carrying the risks of an additional surgical procedure including possible complications from anesthesia, hemorrhage, infection, and post-operative pain. Harvesting periosteum or perichondrium is even more invasive. In vitro culturing of marrow-harvested MSC requires a substantial period of time (2 to 3 weeks) for culturing before the cells can be used in further applications. The additional cell-culturing step renders the method time-consuming, costly, and more highly subject to human error.

In the field of surgery, high energy laser radiation is now well accepted as a surgical tool for cutting, cauterizing, and ablating biological tissue. High energy lasers are now routinely used for vaporizing superficial skin lesions and, and to make deep cuts. For a laser to be suitable for use as a surgical laser, it must provide laser energy at a power sufficient to heat tissue to temperatures over 50 C. Power outputs for surgical lasers vary from 1-5 W for vaporizing superficial tissue, to about 100 W for deep cutting.

In contrast, low level laser therapy involves therapeutic administration of laser energy to a patient at vastly lower power outputs than those used in high energy laser applications, resulting in desirable biostimulatory effects while leaving tissue undamaged. For example, in rat models of myocardial infarction and ischemia-reperfusion injury, low energy laser irradiation reduces infarct size and left ventricular dilation, and enhances angiogenesis in the myocardium. (Yaakboi et al., J. Appl. Physiol. 90, 2411-19 (2001)). Low level laser therapy has been described for treating pain, including headache and muscle pain, and inflammation. The use of low level laser therapy to accelerate bone remodeling and healing of fractures has also been described. (See, e.g., J. Tuner and L. Hode, LOW LEVEL LASER THERAPY, Stockholm:Prima Books, 113-16, 1999, which is incorporated by reference herein).

However, known low level laser therapy methods are circumscribed by setting only certain selected parameters within specified limits. Treatment parameters that can be varied in laser therapy include wavelength, output power (W or mW), dosage (Joules unit area), power density of the light, pulsed or continuous light, pulse frequency, depth of penetration, treatment methodology, treatment density, and total number of treatments over time. Dosage (D) is calculated as:

D=Pxt/A,

where P equals the power output of the laser in watts or milliwatts, t equals the treatment duration in seconds, and A equals the area treated in square centimeters. Known low level laser therapy methods emphasize dosage as the most important treatment parameter, and fix dosages within a range typically of about 0.001 Joules to about 10 Joules/square centimeter. Thus the same dosage can be achieved by compensating a lower power output with a longer treatment time, or a shorter treatment time with a higher power output. Known methods are typically characterized by application of laser energy at a set wavelength using a laser source having a set power output at very low levels of 5 mW to 100 mW, and set treatment times of a few seconds to several minutes to achieve low dosages of at most about 1-10 Joule/cm². However, other parameters can be varied in the use of low level laser therapy yet known low level laser therapy methods have not systematically addressed variation of the multiple other treatment parameters that may contribute to the efficacy of low level laser therapy.

Against this background, a high level of interest remains in finding new and improved therapeutic methods for regenerating bone and cartilage to treat injured or damaged bone and cartilage. A need also remains for improved methods for generating tissue suitable for use in implants to repair damaged bone and cartilage. A need also remains for relatively inexpensive and non-invasive approaches that improve the success of bone and cartilage grafting procedures while also reducing or avoiding the extent of surgery.

### SUMMARY OF THE INVENTION

The low level light therapy methods for regenerating bone and cartilage are based in part on the new and surprising discovery that light energy applied within a certain range of power density (i.e. power per unit area, in watts or milliwatts per cm²) appears to increase the rate at which bone and cartilage tissue regenerates, and also increases the rate at which graft material integrates with surrounding tissue at the graft site. 100171 In one embodiment, there is provided a method directed toward the regeneration of bone and cartilage in a subject in need of such treatment. The method includes delivering a tissue regenerative effective amount of light energy having a wavelength in the visible to near-infrared wavelength range to a site in the bone or cartilage of the subject that includes an area of injury or damage, wherein delivering the tissue regenerative effective amount of light energy includes selecting a power density (mW/cm²) of the light energy to be delivered to the site. Delivering the selected power density to the site includes determining a dosage and a power of the light energy sufficient to deliver the power density to the site.

In preferred embodiments, the power density is at least about 0.01 mW/cm² and less than about 100 mW/cm², including from about 2 mW/cm² to about 20 mW/cm². The light energy preferably has a wavelength of about 630 nm to about 904 nm, including about 780 nm to about 840 nm.

In one embodiment, the method is further directed toward increasing the rate at which graft material implanted at the site of injury or damage integrates with surrounding tissue at the graft site by selecting a power density of light energy to be delivered to the graft site.

In a preferred embodiment, a methods is directed toward placing a light source in contact with a region of skin adjacent the site of injury or damage in bone or cartilage to deliver the tissue regenerative effective amount of light energy to the site by delivering the preselected power density. In addition, to deliver the predetermined power density to the graft site, the method encompasses selecting the dosage and power of the light energy to be sufficient for the light energy to penetrate a thickness of skin and other bodily tissue interposed between the skin surface and the site.

Some preferred methods are further directed toward selecting a dosage and power of a light energy source sufficient for the light energy to traverse the distance between the skin surface and the site.

In one embodiment, the method is directed toward administering an isolated DNA molecule comprising a DNA sequence selected from known isolated gene sequences encoding gene products involved in osteogenesis or chondrogenesis to a subject in need of osteogenesis or chondrogenesis., and delivering a tissue regenerative effective amount of light energy having a wavelength in the visible to near-infrared wavelength range to a site in the bone or cartilage of the subject that includes an area of injury or damage, wherein delivering the tissue regenerative effective amount of light energy includes selecting a power density of light energy to be delivered to the site.

In one embodiment, the method is directed toward administering a recombinant protein encoded by an isolated DNA molecule comprising a DNA sequence selected from known isolated gene sequences encoding gene products involved in osteogenesis or chondrogenesis to a subject in need of osteogenesis or chondrogenesis, and delivering a tissue regenerative effective amount of light energy having a wavelength in the visible to near-infrared wavelength range to a site in the bone or cartilage of the subject that includes an area of injury or damage, wherein delivering the tissue regenerative effective amount of light energy includes selecting a dosage and power of the light energy sufficient to deliver a predetermined power density of light energy to the site.

In one embodiment, the method is directed toward producing cartilage at a cartilage defect site in vivo, the methods including implanting into the defect site a biocompatible, nonliving three-dimensional scaffold structure in combination with periosteal tissue, perichondrial tissue or a combination of periosteal and perichondrial tissues, separately administering into the defect site a preparation of stromal cells for attachment to the scaffold in vivo and for inducing chondrogenesis or migration of stromal cells from the in vivo environment adjacent to the defect site to the scaffold, and delivering a tissue regenerative effective amount of light energy to the defect site wherein delivering a tissue regenerative effective amount of light energy includes selecting a power density (mW/cm²) of the light energy to be delivered to the defect site. The light energy has a wavelength in the visible to near-infrared wavelength range and a power density of at least about 0.01 mW/cm² and not greater than about 100 mW/cm². In an exemplary embodiment, the scaffold is implanted into the defect site and the periosteal or perichondrial tissue is placed on top of and adjacent to the scaffold, or alternatively the periosteal or perichondrial tissue is implanted into the defect site and scaffold is placed on top of and adjacent to the tissue. The scaffold structure is composed of a biodegradable material such as polyglycolic acid, polylactic acid, cat gut sutures, cellulose, nitrocellulose, gelatin, collagen or polyhydroxyalkanoates, or a nonbiodegradable material such as a polyamide, a polyester, a polystyrene, a polypropylene, a polyacrylate, a polyvinyl, a polycarbonate, a polytetrafluoroethylene, polyhydroxyalkanoate, cotton or a cellulose, and may take the form of, for example, a felt or a mesh. The scaffold may be sterilized before implantation, for example with ethylene oxide or by irradiation with an electron beam. In one embodiment, the scaffold includes or is modified to contain at least one substance capable of enhancing the attachment or growth of stromal cells on the scaffold, such as a bioactive agent selected from the group consisting of cellular growth factors, factors that stimulate migration of stromal cells, factors that stimulate chondrogenesis, factors that stimulate matrix deposition, anti-inflammatories, and immunosuppressants. Specific examples of such substances include transforming growth factor-beta (TGF-β), bone morphogenic proteins (BMPs) that stimulate cartilage formation, collagens, elastic fibers, reticular fibers, heparin sulfate, chondroitin-4-sulfate, chondrotin-6-sulfate, dermatan sulfate, keratin sulfate and hyaluronic acid. In one embodiment, the bioactive agent is formulated in a sustained release formulation. In another embodiment, the substance is a biocompatible polymer that forms a composite with the bioactive agent. Specific examples of such biocompatible polymers include polylactic acid, poly(lactic-co-glycolic acid), methylcellulose, hyaluronic acid, and collagen.

In accordance with a preferred embodiment, there is provided a method for forming artificial cartilage. The method comprises delivering a tissue regenerative effective amount of light energy to an in vitro culture comprising a preparation of stromal cells and a substrate for attachment of cells; and culturing the cells in a cell culture chamber for a time sufficient to produce artificial cartilage, wherein delivering a tissue regenerative effective amount of light energy includes delivering light having a wavelength in the visible to near-infrared wavelength range and a power density of at least about 0.01 mW/cm² to the cells during culturing.

In preferred embodiments, the preparation of stromal cells comprises a combination of cells selected from the group consisting of chondrocytes, chondrocyte progenitor cells, fibroblasts, fibroblast-like cells, endothelial cells, pericytes, macrophages, monocytes, leukocytes, plasma cells, mast cells, adipocytes, umbilical cord cells, and bone marrow cells from umbilical cord blood, preferably one or more of chondrocytes, chondrocyte progenitor cells, fibroblasts and fibroblast-like cells. In one embodiment, the stromal cells are mammalian stem cells and the culturing further comprises culturing the stem cells for a time sufficient to allow them to differentiate into chondrocytes. In another embodiment, the stromal cells are mammalian cells other than chondrocytes or chondrocyte stem cells, preferably fibroblasts and/or myocytes, and the culturing further comprises culturing the cells for a time sufficient to allow them to transdifferentiate into chondrocytes.

In preferred embodiments, the method further comprises applying a shear flow stress between about 1 and about 100 dynes/cm² to the cells, preferably between about 1 and about 50 dynes/cm².

In preferred embodiments, the cell culture chamber includes one or more light sources for delivering the tissue regenerative effective amount of light energy.

In one embodiment, the method is directed toward increasing the rate at which an implant or transplant prepared from cartilage cultured on three-dimensional scaffolding in vivo is integrated at a recipient site after transplantation or implantation, by delivering a tissue regenerative effective amount of light energy to the transplantation or implantation site wherein delivering a tissue regenerative effective amount of light energy includes selecting a power density (mW/cm²) of the light energy to be delivered to the culture. The light energy has a wavelength in the visible to near-infrared wavelength range and power density of at least about 0.01 mW/cm² and no greater than about 100 mW/cm².

### BRIEF DESCRIPTION OF THE DRAWINGS

FIG. 1 is a perspective view of a first embodiment of a light therapy device; and

FIG. 2 is a block diagram of a control circuit for the light therapy device, according to one embodiment of the invention.

### DETAILED DESCRIPTION OF THE PREFERRED EMBODIMENT

Preferred Apparatus

The low level light therapy methods for the regeneration of bone and cartilage described herein are practiced and described using, for example, a low level laser therapy apparatus such as that shown and described in U.S. Pat. No. 6,214,035, U.S. Pat. No. 6,267,780, U.S. Pat. No. 6,273,905 and U.S. Pat. No. 6,290,714, which are all herein incorporated by reference together in their entireties with the references contained therein.

A suitable apparatus for the methods for regenerating bone and cartilage herein is a low-level light apparatus including a handheld probe for delivering the light energy. The probe includes a laser source of light energy having a wavelength in the visible to near-infrared wavelength range, i.e. from about 630 nm to about 904 nm. In one embodiment, the probe includes a single laser diode that provides about 25 mW to about 500 mW of total power output, or multiple laser diodes that together are capable of providing at least about 25 mW to about 500 mW of total power output. In other embodiments, the power provided may be more or less than these stated values. The actual power output is preferably variable using a control unit electronically coupled to the probe, so that the power of the light energy emitted can be adjusted in accordance with required power density calculations as described below. In one embodiment, the diodes used are continuously emitting GaAIAs laser diodes having a wavelength of about 830 nm.

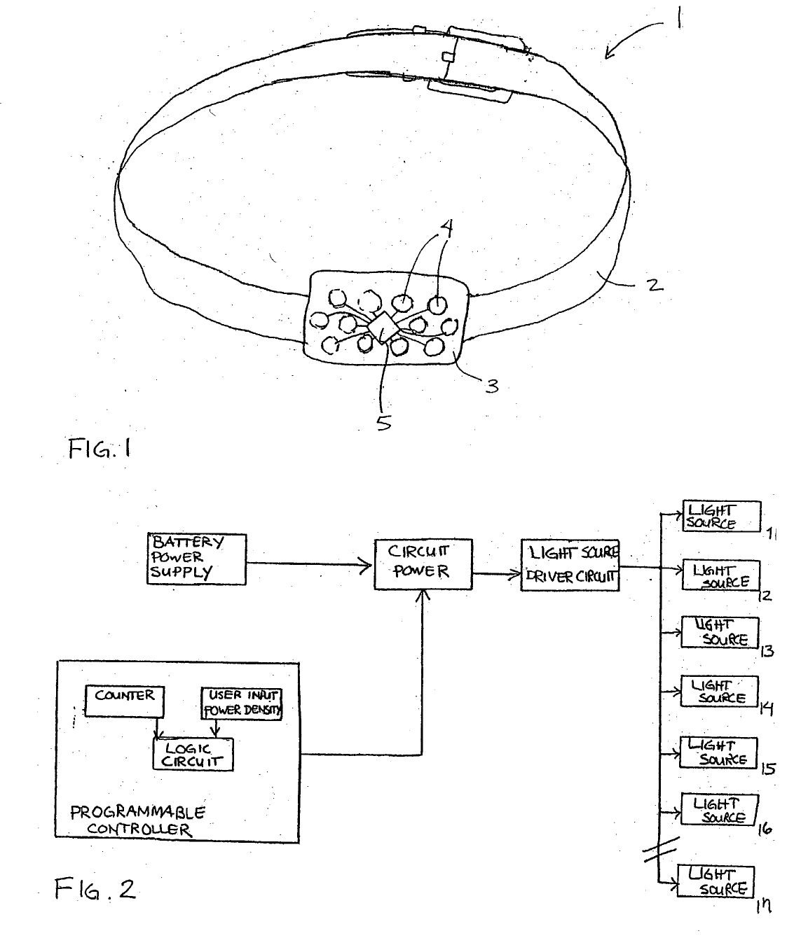

Another suitable light therapy apparatus is that illustrated in FIG. 1. The illustrated device 1 includes a flexible strap 2 with a securing means, the strap adapted for securing the device over an area of the subject's body, one or more light energy sources 4 disposed on the strap 2 or on a plate or enlarged portion of the strap 3, capable of emitting light energy having a wavelength in the visible to near-infrared wavelength range, a power supply operatively coupled to the light source or sources, and a programmable controller 5 operatively coupled to the light source or sources and to the power supply. Based on the surprising discovery that control or selection of power density of light energy is an important factor in determining the efficacy of light energy therapy, the programmable controller is configured to select a predetermined surface power density of the light energy sufficient to deliver a predetermined subsurface power density to a body tissue to be treated beneath the skin surface of the area of the subject's body over which the device is secured.

The light energy source or sources are capable of emitting the light energy at a power sufficient to achieve the predetermined subsurface power density selected by the programmable controller. It is presently believed that tissue will be most effectively treated using subsurface power densities of light of at least about 0.01 mW/cm² and up to about 100 mW/cm², including about 0.05, 0.1, 0.5, 1, 5, 10, 15, 20, 30, 40, 50, 60, 70, 80, and 90 mW/cm². In one embodiment, subsurface power densities of about 0.01 mW/cm² to about 15 mW/cm² are used. To attain subsurface power densities within these stated ranges, taking into account attenuation of the energy as it travels through body tissue and fluids from the surface to the target tissue, surface power densities of from about 100 mW/cm² to about 500 mW/cm² will typically be required, but also possibly to a maximum of about 1000 mW/cm². To achieve such surface power densities, preferred light energy sources, or light energy sources in combination, are capable of emitting light energy having a total power output of at least about 25 mW to about 500 mW, including about 30, 50, 75, 100, 150, 200, 250, 300, and 400 mW, but may also be up to as high as about 1000 mW. It is believed that the subsurface power densities of at least about 0.01 mW/cm² and up to about 100 mW/cm² in terms of the power density of energy that reaches the subsurface tissue are especially effective at producing the desired biostimulative effects on tissue being treated.

The strap is preferably fabricated from an elastomeric material to which is secured any suitable securing means, such as mating Velcro strips, snaps, hooks, buttons, ties, or the like. Alternatively, the strap is a loop of elastomeric material sized appropriately to fit snugly over a particular body part, such as a particular arm or leg joint, or around the chest or hips. The precise configuration of the strap is subject only to the limitation that the strap is capable of maintaining the light energy sources in a select position relative to the particular area of the body or tissue being treated. In an alternative embodiment, a strap is not used and instead the light source or sources are incorporated into or attachable onto a piece of fabric which fits securely over the target body portion thereby holding the light source or sources in proximity to the patient's body for treatment. The fabric used is preferably a stretchable fabric or mesh comprising materials such as Lycra or nylon. The light source or sources are preferably removably attached to the fabric so that they may be placed in the position needed for treatment.

In the exemplary embodiment illustrated in FIG. 1, a light therapy device includes a flexible strap and securing means such as mating Velcro strips configured to secure the device around the body of the subject. The light source or sources are disposed on the strap, and in one embodiment are enclosed in a housing secured to the strap. Alternatively, the light source or sources are embedded in a layer of flexible plastic or fabric that is secured to the strap. In any case, the light sources are preferably secured to the strap so that when the strap is positioned around a body part of the patient, the light sources are positioned so that light energy emitted by the light sources is directed toward the skin surface over which the device is secured. Various strap configurations and spatial distributions of the light energy sources are contemplated so that the device can be adapted to treat different tissues in different areas of the body.

FIG. 2 is a block diagram of a control circuit according to one embodiment of the light therapy device. The programmable controller is configured to select a predetermined surface power density of the light energy sufficient to deliver a predetermined subsurface power density, preferably about 0.01 mW/cm² to about 100 mW/cm², including about 0.01 mW/cm² to about 15 mW/cm² and about 20 mW/cm² to about 50 mW/cm² to the target area. The actual total power output if the light energy sources is variable using the programmable controller so that the power of the light energy emitted can be adjusted in accordance with required surface power energy calculations as described below.

In vitro methods may use similar apparatus to that described above, wherein the apparatus is adapted to irradiate a cell culture in a plate, dish, incubator or other device containing the cell culture. The configuration of the light source is not a critical feature of the methods discussed herein. The light source simply must provide light having the characteristics required by the method of treating the cells.

Definitions and Preferred Parameters

The methods described herein are based primarily on the surprising finding that selecting a power density (i.e. light intensity or power per unit area, in mW/cm²) of light energy from a certain range of power densities appears to be an important factor in determining the efficacy of light therapy in enhancing osteogenesis and chondrogenesis. Thus, in a preferred embodiment, light energy delivered at a power density of at least 0.01 mW/cm² and no more than about 100 mW/cm², irrespective of the power of the light source used and the dosage of the energy used, appears to increase the rate at which bone or cartilage heals after injury or damage, enhances the growth of bone cells and cartilage cells in vitro as well as the rate at which they incorporate onto a scaffold or other support, and also increases the rate at which grafts integrate with surrounding tissue at the recipient graft site. Without being bound by theory, it is believed that independently of the power and dosage of the light energy used, light energy delivered within the specified range of power densities provides the required biostimulative effect on mitochondria to enhance the rate at which cells, particularly osteoblasts, differentiate, grow and migrate to heal injured or damaged bone and cartilage. In the case of grafting, light energy delivered within the specified range of power densities increases the rate at which graft material integrates with surrounding tissue at the recipient graft site.

The term “cartilage” or “cartilage tissue” is used herein as generally recognized in the art and refers to a specialized type of dense connective tissue comprising cells embedded in an extracellular matrix (ECM). While several types of cartilage differing in precise biochemical composition are recognized in the art, the general composition of cartilage comprises chondrocytes surrounded by a dense ECM consisting of collagen, proteoglycans and water. Types of cartilage recognized in the art include, for example, hyaline or articular cartilage such as that found within the joints, fibrous cartilage such as that found within the meniscus and costal regions, and elastic cartilage. Chondrogenesis, i.e. the production or regeneration, of any type of cartilage is intended to fall within the scope of the invention.

The term “chondrocyte progenitor cell” as used herein refers to either (1) a pluripotent, or lineage-uncommitted, progenitor cell, a “stem cell” or “mesenchymal stem cell”, that is potentially capable of an unlimited number of mitotic divisions to either renew its line or to produce progeny cells that will differentiate into chondrocytes; or (2) a lineage-committed progenitor cell produced from the mitotic division of a stem cell which will eventually differentiate into a chondrocyte. Unlike the stem cell from which it is derived, the lineage-committed progenitor is generally considered to be incapable of an unlimited number of mitotic divisions and will eventually differentiate into a chondrocyte.

The term “differentiation” as used herein refers to the process whereby an unspecialized, pluripotent stem cell proceeds through one or more intermediate stage cellular divisions, ultimately producing one or more specialized cell types. Differentiation thus includes the process whereby precursor cells, i.e. uncommitted cell types that precede the fully differentiated forms but may or may not be true stem cells, proceed through intermediate stage cell divisions to ultimately produce specialized cell types. In particular, differentiation encompasses the process whereby mesenchymal stem cells (MSC) are induced to differentiate into the committed cell types comprising bone or cartilage, in vivo or in vitro.

The term “graft” as used herein refers to an amount of viable cells or tissue that is excised from a donor site in a living organism and transferred and inserted at a recipient site in the same or another living organism. A graft may include, for example, precursor cells capable of differentiating into bone or cartilage, MSC, osteoblasts, chondrocytes, chondrocyte progenitor cells, fibroblasts, fibroblast-like cells or cells capable of producing collagen type II and other collagen types, either alone or in various combinations with one another. Donor sources include, for example, bone, cartilage, skin, ligaments, tendons, muscles, placenta, umbilical cord.

The term “integration” as used herein refers to the process whereby a graft material implanted at a graft site is assimilated by the body through migration of undifferentiated cells such as osteoblasts into the graft from surrounding tissue, or from the graft to surrounding tissue, or both, and also through the subsequent differentiation and growth of such undifferentiated cells into differentiated, specialized cell types that restore the injured or damaged bone or cartilage.

The term “regeneration” as used herein refers to the process by which bodily tissue of a certain type needed for the restoration of injured or damaged tissue is regrown from existing viable cells, whether the existing viable cells are cells remaining at a site of damage or injury, or are cells arising from graft material implanted at the site, or both. This term may also be used in connection with processes that occur in vitro or in vivo which generate new tissue from viable cells that are existing at a site or that are placed in an in vitro culture. Osteogenesis refers specifically to the regeneration of bone tissue, and chondrogenesis refers specifically to the regeneration of cartilage tissue. Tissue regeneration as generally used herein is part of a therapeutic strategy for restoring bone or cartilage that is injured or damaged. Injury or damage to bone may arise from various physical traumas including bone fractures sustained in accidental falls, athletic injuries and automobile accidents, or from degenerative conditions of the bone, or genetic or metabolic disorders affecting calcification and bone turnover. For example, such conditions and disorders that can be treated according to the methods of the present invention include but are not limited to dental caries, Paget's disease of bone, osteoporosis, hypocalcemia, hypoparathyroidism, nutritional rickets, metabolic rickets and osteomalacia. Injury or damage to cartilage may arise from any type of physical trauma as described above, or from a degenerative condition of cartilage. Other diseases or conditions or bone or cartilage that can be advantageously treated using the light therapy methods described herein include breakage, depletion or degeneration caused by aging, infectious disease, and repetitive stress. The light therapy methods can be used to treat humans as well as livestock, domestic animals or any other vertebrate species.

The term “regenerative effective” as used herein refers to a characteristic of an amount of light energy which achieves the goal of aiding, promoting or enhancing the process of tissue regeneration, including the generation of new tissue in vitro.

The term “artificial cartilage” as used herein refers to a substance that is substantially similar to or functions substantially as cartilage in the body and comprises cells of the type which comprise natural cartilage. In preferred embodiments, such cells are supported on a substrate or scaffolding.

The terms “scaffolding” and “substrate” as used herein are used interchangeably and generally refer to a material or substance that is used to support and form a base or support network upon which cells attach and grow. In preferred embodiments, artificial cartilage comprises cells cultured upon a substrate. Substrates may be biodegradable, bioabsorbable or non-biodegradable. The terms biodegradable, bioabsorbable or non-biodegradable as used herein are not absolute terms, they include materials that are substantially degradable, bioabsorbable or non-biodegradable.

The terms “growth chamber” and “cell culture chamber” as used herein are used interchangeably and are to be interpreted very broadly to refer to any container or vessel suitable for culturing cells, including, but not limited to, dishes, culture plates (single or multiple well), bioreactors, incubators, and the like.

In preferred embodiments, treatment parameters include the following. Preferred power densities of light at the level of the target cells are at least about 0.01 mW/cm² and up to about 100 mW/cm², including about 0.05, 0.1, 0.5, 1, 5, 10, 15, 20, 30, 40, 50, 60, 70, 80, and 90 mW/cm². To attain subsurface power densities within this preferred range in in vivo methods, one must take into account attenuation of the energy as it travels through body tissue and fluids from the surface to the target tissue, such that surface power densities of from about 25 mW/cm² to about 500 mW/cm² will typically be used, but also possibly to a maximum of about 1000 mW/cm². Such attenuation does not generally need to be accounted for in in vitro methods. To achieve desired power densities, preferred light energy sources, or light energy sources in combination, are capable of emitting light energy having a total power output of at least about 1 mW to about 500 mW, including about 5, 10, 15, 20, 30, 50, 75, 100, 150, 200, 250, 300, and 400 mW, but may also be up to as high as about 1000 mW or below 1 mW. Preferably the light energy used for treatment has a wavelength in the visible to near-infrared wavelength range, i.e., from about 630 to about 904 nm, preferably about 780 nm to about 840 nm, including about 790, 800, 810, 820, and 830 nm.

In preferred embodiments, the light source used in the light therapy is a coherent source (i.e. a laser), and/or the light is substantially monochromatic (i.e. one wavelength or a very narrow band of wavelengths).

In preferred embodiments, the treatment proceeds continuously for a period of about 30 seconds to about 2 hours, more preferably for a period of about 1 to 20 minutes. The treatment may be terminated after one treatment period, or the treatment may be repeated with preferably about 1 to 2 days passing between treatments. The length of treatment time and frequency of treatment periods can be varied as needed to achieve the desired result.

During the treatment, the light energy may be continuously provided, or it may be pulsed. If the light is pulsed, the pulses are preferably at least about 10 ns long, including about 100 ns, 1 ms, 10 ms, and 100 ms, and occur at a frequency of up to about 1 kHz, including about 1 Hz, 10 Hz, 50 Hz, 100 Hz, 250 Hz, 500 Hz, and 750 Hz.

Preferred in vivo Methods

In a preferred embodiment, methods directed toward the regeneration of bone and cartilage in a subject in need of such treatment include delivering a tissue regenerative effective amount of light energy having a wavelength in the visible to near-infrared wavelength range to a site in the bone or cartilage of the subject that includes an area of injury or damage, wherein delivering the tissue regenerative effective amount of light energy includes selecting a dosage and power of the light energy sufficient to deliver a predetermined power density of light energy to the site. The methods also are directed toward increasing the rate at which graft material implanted at the site of injury or damage integrates with surrounding tissue at the graft site.

One method for regenerating bone or cartilage in a subject in need of such treatment involves delivering a tissue regenerative effective amount of light energy having a wavelength in the visible to near-infrared wavelength range to a site in the bone or cartilage of the subject that includes an area of injury or damage. Delivering the tissue regenerative effective amount of light energy includes selecting a power density to be delivered to the site. Selecting the power density to be delivered to the site includes selecting a dosage and power of the light energy sufficient to deliver the selected power density of light energy to the site. The power density and other parameters are preferably as noted above.

It is understood that the power density selected will be dependent upon a variety of factors including the age, gender, health, and weight of the recipient, type of concurrent treatment, if any, frequency of treatment, and the precise nature of the effect desired.

To deliver the selected power density at the site of injury or damage, a required, relatively greater surface power density of the light energy is calculated taking into account attenuation of the light energy as it travels through various tissues including skin, muscle and fat tissue. Factors known to affect penetration and to be taken into account in the calculation include skin pigmentation, and the location of the site being treated, particularly the depth of the site being treated relative to the surface. For example, to obtain a desired power density of about 10 mW/cm² at the site of injury or damage at a depth of 3 cm below the skin surface may require a surface power density of 400 mW/cm². The higher the level of skin pigmentation, the higher the required surface power density to deliver a predetermined power density of light energy to a subsurface site of injury or damage.

To treat a patient in need of bone or cartilage regeneration, the light source is placed in contact with or nearly in contact with a region of skin, for example adjacent a bone fracture that has been identified and located using standard medical imaging techniques such as X-ray. The power density calculation takes into account factors including the location within the body of the bone or cartilage being treated, the extent and type of intervening body tissue such as fat and muscle between the skin surface and the site of injury or damage, skin coloration, distance to the damaged or injured site, etc. that affect penetration and thus power density actually received at the site of injury or damage. Power of the light source being used and the surface area treated are accordingly adjusted to obtain a surface power density sufficient to deliver the predetermined power density of light energy to the subsurface site of injury or damage. The light energy source is energized and the selected power density of light energy delivered to the injured or damaged bone or cartilage.

Within the described preferred range, the precise power density selected for treating the patient is determined according to the judgment of a trained healthcare provider, such as a physician or technician, and depends on a number of factors, including the specific wavelength or light selected, and clinical factors such as the extent and type of injury or damage being treated (i.e. fracture, surgical intervention, degenerative bone condition), the clinical condition of the subject including the location of the bone or cartilage affected and the type of bone, if any, affected, and the like. Similarly, it should be understood that the power density of light energy might be adjusted to be combined with any other therapeutic agent or agents, especially pharmaceutical anti-inflammatory agents to achieve the desired biological effect. The selected power density will again depend on a number of factors, including the specific light energy wavelength chosen, the individual additional therapeutic agent or agents chosen, and the clinical condition of the subject.

Applying the methods to conventional bone grafting procedures in a preferred embodiment, a bone defect site (recipient graft site) is surgically exposed and filled with either autologous or allograft material. The graft material used may be live active bone tissue, such as fresh autogenous cancellous bone and marrow including osteoblasts, DFDBA, or autogenous cortical bone chips. A barrier material such as a PTFE membrane can be used to cover the graft site.

Alternatively, graft material may instead comprise or derive from, stem cells or precursor cells such as MSC that are isolated from periosteum, perichrondrium or marrow by culturing in vitro or in vivo. For example, periosteum or marrow can be used as an in vivo source of precursor cells having osteogenic potential. Periosteum, perichondrium or marrow can be used as an in vivo source of cartilage precursor cells. In vitro culturing can be used to isolate and amplify stems cells from harvested periosteum, perichondrium or marrow, and then the isolated, amplified stem cells introduced into a recipient at the site of injury or damage. For example, MSC can be isolated and amplified from marrow using in vivo culturing and then introduced at the site of injury or damage. The wound is then surgically closed and low level light energy is then applied to skin adjacent the graft site, at the power density predetermined in accordance with the judgment of a trained health care provider or light therapy technician, according to the methods described herein.

With particular reference to methods for cartilage replacement, the light therapy methods as described herein are also advantageously used in combination with methods using a three-dimensional scaffold as described in U.S. Pat. No. 5,842,477 (which is herein incorporated by reference) for making or repairing cartilage in vivo. The methods include implanting into a patient, at a site of cartilage damage or loss, a biocompatible, non-living three-dimensional scaffold or framework structure in combination with periosteal or perichondrial tissue that can be used to hold the scaffold in place and to provide a source of chondrocyte progenitor cells, chondrocytes and other stromal cells for attachment to the scaffold in vivo. In addition, a preparation of cells that includes any or all of chondrocytes, chondrocyte progenitor cells and other stromal cells is administered, either before, during or after implantation of the scaffold and the periosteal/perichondrial tissue, or after implantation of the scaffold but before implantation of the periosteal/perichondrial tissue. The cells are administered directly into the site of the implant in vivo to promote chondrogenesis and the production of factors that induce the migration of chondrocytes, progenitor cells and other stromal cells from the adjacent in vivo environment into the scaffold for the production of new cartilage at the site of implantation. After implantation of the scaffold and periosteal/perichondrial tissue, low level light energy in accordance with the methods herein described is applied to the site. More specifically, as a preliminary step, light energy having a power density of at least about 0.01 mW/cm² up to about 100 mW/cm² may be applied directly to the implant before surgical closing of the defect site. After surgical closing of the implant site, light energy having a power density sufficient to provide a power density of at least about 0.01 mW/cm² up to about 100 mW/cm² at the implant site is applied to a skin surface adjacent the implant site to promote chondrogenesis. The preparation of stromal cells seeded in combination with the scaffold and periosteal/perichondrial tissue provides a ready source of chondrocytes and other stromal cells which produce biological factors that together with the application of light energy of a select power density promote chondrogenesis and the migration of stromal cells from, e.g., the periosteal/perichondrial tissue to the scaffold for attachment and/or differentiation thereon. The stromal cell preparation also provides a direct source of stromal cells, e.g., chondrocytes and/or progenitor cells, that are capable of migrating into the scaffold and attaching thereto. The stromal cells in the scaffold, whether derived from the periosteal/perichondrial tissue, from the exogenous stromal cell preparation or from the in vivo environment adjacent to the implant site, grow on the scaffold to form a cellular matrix and provide the support, growth factors and regulatory factors required for cartilage formation at a cartilage defect site in vivo. Without being bound by theory, it is believed that the application of light energy within the stated power density range has a biostimulatory effect on mitochondria in the stromal cells in the scaffold, and also on surrounding cells, thereby enhancing stromal cell function and enhancing the in vivo formation of new cartilage at the implant site.

Gene Therapy

The light therapy methods as described herein can also be advantageously used in combination with gene therapy to regenerate bone and cartilage. For example, as described in U.S. Pat. No. 6,143,878 (which is herein incorporated by reference herein in its entirety), DNA sequences of the Sox-9 and SOX-9 genes have been isolated and identified and the gene products thereof linked to the processes of osteogenesis and chondrogenesis. Such sequences, or recombinant proteins encoded by and generated from gene sequences such as these or those having similar biological effects, can be used in combination with the light therapy methods described herein to regenerate bone or cartilage. More specifically, an isolated DNA molecule including such sequences, or the recombinant proteins, or both in combination, can be administered to a patient in need of bone or cartilage regeneration, followed by light therapy as described above to facilitate the process of bone or cartilage regeneration.

Therefore, in another aspect, the present methods include administering an isolated DNA molecule comprising a DNA sequence selected from known isolated gene sequences encoding gene products involved in osteogenesis or chondrogenesis to a subject in need of osteogenesis or chondrogenesis, and delivering a tissue regenerative effective amount of light energy having a wavelength in the visible to near-infrared wavelength range to a site in the bone or cartilage of the subject that includes an area of injury or damage, wherein delivering the tissue regenerative effective amount of light energy includes selecting a dosage and power of the light energy sufficient to deliver a predetermined power density of light energy to the site, as discussed in greater detail herein above.

In another embodiment, the present methods include administering a recombinant protein encoded by an isolated DNA molecule comprising a DNA sequence selected from known isolated gene sequences encoding gene products involved in osteogenesis or chondrogenesis, to a subject in need of osteogenesis or chondrogenesis, and delivering a tissue regenerative effective amount of light energy having a wavelength in the visible to near-infrared wavelength range to a site in the bone or cartilage of the subject that includes an area of injury or damage, wherein delivering the tissue regenerative effective amount of light energy includes selecting a dosage and power of the light energy sufficient to deliver a predetermined power density of light energy to the site as discussed in greater detail herein above. The recombinant protein may be prepared by known molecular biological techniques including ligating a DNA sequence encoding a recombinant protein of the DNA sequence selected from known isolated gene sequences encoding gene products involved in osteogenesis or chondrogenesis, or a biological fragment thereof, into a suitable expression vector to form an expression construct; transfecting the expression construct into a suitable host cell; expressing the recombinant protein; and isolating the recombinant protein. For example, the recombinant protein can be prepared by a person skilled in the art using standard protocols such as those described in Sambrook et al., MOLECULAR CLONING: A LABORATORY MANUAL, Cold Spring Harbour Laboratory Press: New York, 1989, wherein the vector may be a prokaryotic or a eukaryotic expression vector, and the host cell for expression of the recombinant protein can be a prokaryote or eukaryote.

In yet another embodiment, the methods include a method of regeneration of bone or cartilage by administration of a suitable DNA molecule or protein as explained above to a subject suffering from bone or cartilage deficiency. The DNA molecule or protein may be injected directly into joint tissue such as knees, knuckles, elbows, ankles or ligaments. The DNA molecule or protein can also be administered by systemic injection, surgical implantation, instillation or by any other means. Such genetic therapy may also be used in combination with local application by injection, surgical implantation, instillation or any other means, of cells responsive to the DNA molecule or protein The genetic therapy may also be used in combination with local application by injection, surgical implantation, instillation or any other means, of other therapeutic agents that promote osteogenesis or chondrogenesis.

In vitro Methods

The seeding and culturing of tissue for use in replacement therapy is known in the art. For example, methods for culturing cartilage in vitro on biocompatible three-dimensional scaffolding are described in U.S. Pat. No. 5,902,741, U.S. Pat. No. 6,060,306, and U.S. Pat. No. 5,928,945, all of which are incorporated herein by reference in their entireties. In preferred embodiments, cells are seeded and cultured in a dynamic environment. Cells cultured in dynamic environments are more likely to tolerate the physiological conditions which exist in the human body once they are implanted because culturing conditions of periodic or continuous fluid flow and pressure more closely resemble the conditions under which chondrocytes are cultured in the human body, resulting in the formation of a tissue-engineered cartilage construct that possesses physical and biochemical properties more similar to that of native cartilage. The light therapy methods as described herein are advantageously used in combination with such dynamic in vitro tissue culture methods and also static tissue culture methods.

In a preferred culture method, stromal cells are inoculated and grown on the biocompatible three-dimensional scaffold or framework, preferably in the presence of a growth factor such as TGF-β, and light energy is applied to the three-dimensional cell culture in vitro, using preferred parameters including power density as discussed above. Because the light energy is applied directly to the cell culture in vitro and does not travel through intervening body tissue, the power density selected to be delivered to the cell is generally equal to the power density of the light energy as it is emitted from the light apparatus, although there may be lenses, filters or dispersion gratings or the like used. Applying light energy to the in vitro culture at a select power density enhances the formation of three-dimensional cartilage cultures in vitro on scaffolding.

For example, cartilage is prepared in vitro using three-dimensional culture methods such as those described in U.S. Pat. No. 5,902,741. More specifically, stromal cells, such as chondrocytes, progenitor-chondrocytes, fibroblasts and/or fibroblast-like cells are grown on a three-dimensional scaffold or framework in vitro under conditions that enhance the formation of cartilage in culture. A variety of biodegradable and nonbiodegradable matrices treated with sterilizing agents and/or procedures can be used as the scaffold in accordance with the preferred embodiments. The selected power density of light energy applied to the in vitro culture can be varied within the specified preferred range while taking into account other culture variable such as pressure and the addition of growth factors. After in vitro formation of cartilage sufficient for implantation, the cartilage is transplanted or implanted in vivo at a cartilage defect site and the site of implantation treated with light energy according to the methods described supra, to enhance chondrogenesis and the rate at which the implant is integrated with surrounding tissue at the recipient site.

In another embodiment, shear flow stress and low level light therapy as described herein are both applied to chondrocytes, chondrocyte stem cells or certain other cell types in culture to produce artificial cartilage for surgical repair of damaged cartilage. One preferred method of culture is as described in U.S. Pat. No. 5,928,945, to which can be added the light treatment conditions disclosed herein. Cultured chondrocytes do not align under shear flow stress, and application of shear flow stress to cultured chondrocytes enhances maintenance of chondrocyte phenotype, resulting in enhanced type II collagen deposition in the chondrocytes.

In a preferred embodiment, the present methods include applying low level light therapy to cells in culture in a bioreactor for producing artificial cartilage. The bioreactor includes a growth chamber for housing cultured mammalian cells, a substrate for attachment of the cells, and means for applying shear flow stress at a level between about 1 and about 100 dynes/cm². In one embodiment, the bioreactor is capable of applying shear flow stress at a level between about 1 and about 50 dynes/cm². To generate the shear flow stress, a pump system including a reservoir, a pump, and interconnecting tubing is arranged to allow continuous flow of liquid growth medium from the reservoir, through the growth chamber, and back to the reservoir, in response to force applied by the pump. Cells are cultured on a substrate in the bioreactor which is, for example, a scaffold that supports growth of a 3-dimensional cell culture. The scaffold is preferably bioabsorbable, or is a nonporous surface that supports the growth of cultured cells in a monolayer. The nonporous surface can be, for example, the smooth surface of a rotatable drum, a rotatable disc, or a static plate. When a drum or disc is used, shear flow stress is generated by movement, i.e., rotation, of the drum or disc through the liquid culture medium.

In a preferred embodiment, a method for producing artificial cartilage includes the following: providing a growth chamber containing a substrate for attachment of cells; bathing the substrate with a liquid growth medium; inoculating into the medium chondrocytes, chondrocyte stem cells, or cells that transdifferentiate into a chondrocyte phenotype; allowing the cells to attach to the substrate; applying and maintaining shear flow stress between about 1 and about 100 dynes/cm² to the cells, preferably between about 1 and about 50 dynes/cm²; culturing the shear flow stressed cells for a time sufficient to produce artificial cartilage and during culturing delivering a tissue regenerative effective amount of light energy having a wavelength in the visible to near-infrared wavelength range to the cells on the substrate, wherein delivering a tissue regenerative effective amount of light energy includes selecting a dosage and power of the light energy sufficient to deliver a predetermined power density of light energy to the cells on the substrate. The predetermined power density is preferably a power density of at least about 0.01 mW/cm², and in one embodiment is a power density selected from the range of about 0.01 mW/cm² to about 100 mW/cm². In an exemplary embodiment, the predetermined power density is selected from the range of about 2 mW/cm² to about 20 mW/cm². The light energy has a wavelength of about 630 nm to about 904 nm, preferably about 780 nm-840 nm. The substrate is preferably bioabsorbable, or is a nonporous surface that supports the growth of cultured cells in a monolayer such as the smooth surface of a rotatable drum, a rotatable disc, or a static plate.

In another embodiment, there is a method for inducing differentiation of stem cells into chondrocytes. The stem cell differentiation method includes: providing a growth chamber containing a substrate for the attachment of cells; bathing the substrate with a liquid growth medium; inoculating into the medium mammalian stem cells; allowing the stem cells to attach to the substrate; applying and maintaining shear flow stress between about 1 and about 100 dynes/cm², including about 1 and about 50 dynes/cm² to the stem cells; culturing the stem cells for a time sufficient to allow them to differentiate into chondrocytes; and during culturing delivering a tissue regenerative effective amount of light energy having a wavelength in the visible to near-infrared wavelength range to the cells on the substrate, wherein delivering a tissue regenerative effective amount of light energy includes selecting a dosage and power of the light energy sufficient to deliver a predetermined power density of light energy to the cells on the substrate. The method parameters for light treatment are preferably those described supra. The substrate is preferably bioabsorbable, or is a nonporous surface that supports the growth of cultured cells in a monolayer such as the smooth surface of a rotatable drum, a rotatable disc, or a static plate.

In another embodiment, there is a method for inducing transdifferentiation of cultured cells into chondrocytes including: providing a growth chamber containing a substrate for attachment of cells; bathing the substrate with a liquid growth medium; inoculating into the medium mammalian cells other than chondrocytes or chondrocyte stem cells; allowing the cells to attach to the substrate; applying and maintaining shear flow stress between about 1 and about 100 dynes/cm², preferably between about 1 and about 50 dynes/cm², to the cells; culturing cells for a time sufficient to allow them to transdifferentiate into chondrocytes; and during culturing delivering a tissue regenerative effective amount of light energy having a wavelength in the visible to near-infrared wavelength range to the cells on the substrate, wherein delivering a tissue regenerative effective amount of light energy includes selecting a dosage and power of the light energy sufficient to deliver a predetermined power density of light energy to the cells on the substrate. The method parameters for light treatment are preferably as described above. Preferred nonchondrocyte cell types for use in this transdifferentiation method are fibroblasts and myocytes.

In another embodiment, the present methods include a method for seeding and culturing tissue in a cell culture chamber as described in U.S. Pat. No. 6,060,306, including disposing within the chamber a porous, three-dimensional substrate configured and dimensioned to seal with the chamber, the substrate configured to facilitate three-dimensional tissue growth on the substrate and including a three-dimensional framework having interstitial spaces bridgeable by cells; exposing the substrate in the chamber to a flow of fluid media for seeding and culturing; alternatingly creating a pressure differential across the substrate during seeding and culturing to force substantially all flow of the fluid media within the chamber through the substrate and facilitate exposure of the substrate to the fluid media, and during culturing delivering a tissue regenerative effective amount of light energy having a wavelength in the visible to near-infrared wavelength range to the cells on the substrate, wherein delivering a tissue regenerative effective amount of light energy includes selecting a dosage and power of the light energy sufficient to deliver a predetermined power density of light energy to the cells on the substrate. The method parameters for light treatment are preferably as described above. The step of creating pressure is achieved, for example, by placing the substrate in a fluid-filled chamber, the chamber filled with fluid media for seeding and culturing; and applying a pressure differential to the fluid media across the substrate so that fluid media is forced through the substrate. This is accomplished, for example, using a pump. Various known simple mechanical systems are suitable for delivering and maintaining the fluid flow.

Therefore, in another aspect, the present methods include enhancing the in vitro formation of cartilage in culture on three-dimensional scaffolding by applying light energy, preferably light energy having a wavelength in the visible to near-infrared wavelength range at a power density of at least about 0.01 mW/cm² and no greater than about 100 mW/cm² to the in vitro cartilage culture. The methods also encompass increasing the rate at which an implant or transplant prepared from cartilage cultured on three-dimensional scaffolding in vitro is integrated at a recipient site after transplantation or implantation. Thus, to enhance the rate of integration of such a cartilage transplant or implant, the methods include applying light to a region of skin adjacent the site of transplantation or implantation of the cultured cartilage.

### EXAMPLE

An in vitro experiment was done to demonstrate the effect of light treatment on fibroblasts. Normal Human Dermal Fibroblast (NHDF) cells were obtained cryopreserved through Clonetics (Baltimore, Md.). NHDF cells were thawed and cultured in flasks with reagents provided with the cells, following the manufacturer's instructions. The cells were then plated into 96 well plates (black plastic with clear bottoms, Becton Dickinson, Franklin Lakes N.J.) at a density of 1000 cells per well, using the same medium without serum for 24 hours prior to lasing. This process of “serum starvation” was intended to simulate an “injury” to the cells. Assays were then performed to determine how much the laser treatments accelerated growth following the injury.

A Photo Dosing Assembly (PDA) was used to provide precisely metered doses of laser light to the NHDF cells in the 96 well plate. The PDA consisted of a Nikon Diaphot inverted microscope (Nikon, Melville, N.Y.) with a LUDL motorized x,y,z stage (Ludl Electronic Products, Hawthorne, N.Y.). An 808 nm laser was routed into the rear epi-fluorescent port on the microscope using a custom designed adapter and a fiber optic cable. Diffusing lenses were mounted in the path of the beam to create a “speckled” pattern, which was intended to mimic in vivo conditions after a laser beam passed through human skin. The beam diverged to a 25 mm diameter circle when it reached the bottom of the 96 well plate. This dimension was chosen so that a cluster of four adjacent wells could be lased at the same time. Cells were plated in a pattern such that a total of 12 clusters could be lased per 96 well plate. Stage positioning was controlled by a Silicon Graphics workstation and laser timing was performed by hand using a digital timer. The measured average, continuous, power density passing through the plate for the NHDF cells was 50 mW/cm².

The assay used to quantify the redox state within the NHDF cells was the alamarBlue assay (Biosource, Camarillo, Calif.). The internal environment of a proliferating cell is more reduced than that of a non-proliferating cell. Specifically, the ratios of NADPH/NADP, FADH/FAD, FMNH/FMN and NADH/NAD, increase during proliferation. Laser irradiation is also thought to have an effect on these ratios. Compounds such as alamarBlue are reduced by these metabolic intermediates and can be used to monitor cellular states. The oxidization of alamarBlue is accompanied by a measurable shift in color. The in its unoxidized state, alamarBlue appears blue. When oxidized, the color changes to red. To quantify this shift, a 340 PC microplate reading spectrophotometer (Molecular Devices, Sunnyvale, Calif.) was used to measure the absorbance of a well containing NHDF cells, media and alamarBlue diluted 10% v/v. The absorbance of each well was measured at 570 nm and 600 nm and the percent reduction of alamarBlue was calculated using an equation provided by the manufacturer.

The percent reduction of alamarBlue described above was used to compare NHDF culture wells that had been lased for different durations (0, 6, 9, 20, 25 minutes) with 50 mW/cm² at a wavelength of 808 nm. alamarBlue was added immediately after lasing and the absorbance was measured 7.5 hours later. The average measured values for percent reduction were: 24.1% for the control group that was not lased, 25.9% for an energy dose of 3J/cm² (an increase of 7.2% over the control, p=0.17 using the t-test for significance), 29.5% for 27J/cm² (22.2% increase, p=0.004), 37.3% for 60J/cm² (54.5% increase, p=0.002), and 44.5% for 75J/cm² (84.4% increase, p=0.0002). These alamarBlue results show a positive effect of infra-red laser treatment on the cells that increases with increasing energy dose.

A more reduced state within the cell is considered to be an indication that the cell is viable, healthy and proliferating. These results are novel and significant in that they show the positive effects of infra-red laser irradiation on cellular metabolism in in-vitro fibroblast cell cultures.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

What is claimed is:

1. A method for the regeneration of bone or cartilage in a subject in need of such treatment, said method comprising delivering a tissue regenerative effective amount of light energy having a wavelength in the visible to near-infrared wavelength range to a site in the bone or cartilage of the subject that includes an area of injury or damage wherein delivering the tissue regenerative effective amount of light energy comprises selecting a dosage and power of the light energy sufficient to deliver a predetermined power density of light energy to the site of at least about 0.01 mW/cm².

2. A method in accordance with claim 1 wherein the selected power density is a power density selected from the range of about 0.01 mW/cm² to about 100 mW/cm².

3. A method in accordance with claim 1 wherein the light energy has a wavelength of about 630 nm to about 904 nm.

4. A method in accordance with claim 1, wherein the light energy has a wavelength of about 780 nm to about 840 nm.

5. A method in accordance with claim 1, wherein the light is delivered in pulses at a frequency of about 1 Hz to about 1 kHz.

6. A method in accordance with claim 1 wherein delivering a tissue regenerative effective amount of light energy to the site comprises placing a light source in contact with a region of skin adjacent the site of bone or cartilage including the area of injury or damage.

7. A method in accordance with claim 1 wherein selecting a dosage and power of the light energy sufficient to deliver a predetermined power density of light energy to the site comprises selecting the dosage and power of the light sufficient for the light energy to penetrate body tissue interposed between the skin surface and the site of injury or damage.

8. A method for treating injury or damage of bone or cartilage comprising administering an isolated DNA molecule comprising a DNA sequence selected from known isolated gene sequences encoding gene products involved in osteogenesis or chondrogenesis to a subject in need osteogenesis or chondrogenesis, and delivering a tissue regenerative effective amount of light energy having a wavelength in the visible to near-infrared wavelength range to a site in the bone or cartilage of the subject that includes an area of injury or damage, wherein delivering the tissue regenerative effective amount of light energy includes selecting a power density of light energy to be delivered to the site of at least about 0.01 mW/cm².

9. A method in accordance with claim 8 wherein the selected power density is a power density selected from the range of about 0.01 mW/cm² to about 100 mW/cm².

10. A method in accordance with claim 8 wherein the light energy has a wavelength of about 630 nm to about 904 nm.

11. A method for treating injury or damage of bone or cartilage comprising administering a recombinant protein encoded by an isolated DNA molecule comprising a DNA sequence selected from known isolated gene sequences encoding gene products involved in osteogenesis or chondrogenesis to a subject in need of osteogenesis or chondrogenesis, and delivering a tissue regenerative effective amount of light energy having a wavelength in the visible to near-infrared wavelength range to a site in the bone or cartilage of the subject that includes an area of injury or damage, wherein delivering the tissue regenerative effective amount of light energy includes selecting a power density of light energy to be delivered to the site of at least about 0.01 mW/cm².

12. A method in accordance with claim 11 wherein the selected power density is a power density selected from the range of about 0.01 mW/cm² to about 100 mW/cm².

13. A method in accordance with claim 11 wherein the light energy has a wavelength of about 630 nm to about 904 nm.

14. A method for increasing the rate at which an implant or transplant prepared from cartilage cultured on three-dimensional scaffolding in vitro is integrated at a recipient site after transplantation or implantation, by delivering a tissue regenerative effective amount of light energy to the transplantation or implantation site wherein delivering a tissue regenerative effective amount of light energy includes selecting a power density (mW/cm²) of the light energy to be delivered to the culture of at least about 0.01 mW/cm².

15. A method in accordance with claim 14 wherein the selected power density is a power density selected from the range of about 0.01 mW/cm² to about 100 mW/cm².

16. A method of producing cartilage at a cartilage defect site in vivo comprising:
implanting into the defect site a biocompatible, non-living three-dimensional scaffold structure in combination with periosteal tissue, perichondrial tissue or a combination of periosteal and perichondrial tissues;
separately administering into the defect site a preparation of stromal cells for attachment to the scaffold in vivo and for inducing chondrogenesis or migration of stromal cells from the in vivo environment adjacent to the defect site to the scaffold; and
delivering a tissue regenerative effective amount of light energy to the defect site wherein delivering a tissue regenerative effective amount of light energy includes selecting a power density (mW/cm²) of the light energy to be delivered to the culture of at least about 0.01 mW/cm².

17. A method in accordance with claim 16 wherein the selected power density is a power density selected from the range of about 0.01 mW/cm² to about 100 mW/cm².

18. The method of claim 16, wherein the scaffold is implanted into the defect site and the periosteal or perichondrial tissue is placed on top of and adjacent to the scaffold.

19. The method of claim 16, wherein the periosteal or perichondrial tissue is implanted into the defect site and the scaffold is placed on top of and adjacent to the tissue.

20. The method of claim 16, wherein the periosteal or perichondrial tissue is situated with respect to the scaffold such that stromal cells from the tissue can migrate from the tissue to the scaffold.

21. The method of claim 16, wherein the periosteal tissue or perichondrial tissue is situated with respect to the scaffold such that the cambium layer of the tissue faces the scaffold.

22. The method of claim 16, wherein the preparation of stromal cells is administered prior to, during or after implantation of the scaffold structure.

23. The method of claim 16, wherein the preparation of stromal cells is administered prior to, during or after implantation of the periosteal or perichondrial tissue.

24. The method of claim 16, wherein the preparation of stromal cells is physically placed between the scaffold and the periosteal or perichondrial tissue.

25. The method of claim 16, wherein the scaffold structure is composed of a biodegradable material.

26. The method of claim 25, wherein the biodegradable material is polyglycolic acid, polylactic acid, cat gut sutures, cellulose, nitrocellulose, gelatin, collagen, or polyhydroxyalkanoates.

27. The method of claim 16, wherein the scaffold structure is composed of a non-biodegradable material.

28. The method of claim 27, wherein the non-biodegradable material is a polyamide, a polyester, a polystyrene, a polypropylene, a polyacrylate, a polyvinyl, a polycarbonate, a polytetrafluoroethylene, polyhydroxylalkanoate, cotton or a cellulose.

29. The method of claim 16, wherein the scaffold is a felt or mesh.

30. The method of claim 16, wherein the scaffold is treated with ethylene oxide or electron beam prior to implantation.

31. The method of claim 16, wherein the scaffold comprises or is modified to contain at least one substance capable of enhancing the attachment or growth of stromal cells on the scaffold.

32. The method of claim 31, wherein the substance is a bioactive agent selected from the group consisting of cellular growth factors, factors that stimulate migration of stromal cells, factors that stimulate chondrogenesis, factors that stimulate matrix deposition, anti-inflammatories, and immunosuppressants.

33. The method of claim 32, wherein the bioactive agent is a transforming growth factor-beta or a bone morphogenetic protein that stimulates cartilage formation.

34. The method of claim 32, wherein the bioactive agent further comprises a sustained release formulation.

35. The method of claim 34 further comprising a biocompatible polymer which forms a composite with the bioactive agent.

36. The method of claim 35, wherein the biocompatible polymer is selected from the group consisting of polylactic acid, poly(lactic-co-glycolic acid), methylcellulose, hyaluronic acid, and collagen.

37. The method of claim 31, wherein the substance is selected from the group consisting of collagens, elastic fibers, reticular fibers, heparin sulfate, chondroitin-4-sulfate, chondroitin-6-sulfate, dermatan sulfate, keratin sulfate and hyaluronic acid.

38. The method of claim 16, further comprising the step of administering to the defect site at least one substance capable of enhancing the attachment or growth of stromal cells on the scaffold.

39. The method of claim 38, wherein the substance is a bioactive agent selected from the group consisting of cellular growth factors, factors that stimulate migration of stromal cells, factors that stimulate chondrogenesis, factors that stimulate chondrogenesis, factors that stimulate matrix deposition, anti-inflammatories, and immunosuppressants.

40. The method of claim 39, wherein the substance is a transforming growth factor-beta.

41. The method of claim 39, wherein the bioactive agent is a bone morphogenetic protein that stimulates cartilage formation.

42. The method of claim 16, wherein the periosteal or perichondrial tissue is autologous to the defect site.

43. The method of claim 16, wherein the preparation of stromal cells comprises chondrocytes, chondrocyte progenitor cells, fibroblasts and/or fibroblast-like cells.

44. The method of claim 16, wherein the preparation of stromal cells comprises a combination of cells selected from the group consisting of chondrocytes, chondrocyte progenitor cells, fibroblasts, fibroblast-like cells, endothelial cells, pericytes, macrophages, monocytes, leukocytes, plasma cells, mast cells, adipocytes, umbilical cord cells, and bone marrow cells from umbilical cord blood.

45. The method of claim 16, wherein the preparation of stromal cells comprises at least one bioactive agent.

46. The method of claim 45, wherein the bioactive agent is selected from the group consisting of cellular growth factors, factors that stimulate migration of stromal cells, factors that stimulate chondrogenesis, factors that stimulate matrix deposition, anti-inflammatories, and immunosuppressants.

47. The method of claim 46, wherein the bioactive agent is a transforming growth factor-beta or a bone morphogenetic protein that stimulates cartilage formation.

48. The method of claim 16, wherein the stromal cells of the preparation are genetically engineered to produce at least one bioactive agent.

49. The method of claim 48, wherein the bioactive agent is selected from the group consisting of cellular growth factors, factors that stimulate migration of stromal cells, factors that stimulate chondrogenesis, factors that stimulate matrix deposition, anti-inflammatories, and immunosuppressants.

50. The method of claim 16, wherein the stromal cells of the preparation are genetically engineered to express a gene that is deficiently expressed in vivo.

51. The method of claim 16, wherein the stromal cells of the preparation are genetically engineered to prevent or reduce the expression of a gene expressed by the stromal cells.

52. The method of claim 16, wherein the cartilage defect site is treated to degrade the existing cartilage at the site.

53. The method of claim 52, wherein the treatment is selected from the group consisting of enzyme treatment, abrasion, debridement, shaving, and microdrilling.

54. The method of claim 53, wherein the enzyme treatment utilizes at least one enzyme selected from the group consisting of trypsin, chymotrypsin, collagenase, elastase, hyaluronidase, DNAase, pronase and chondroitinase.

55. The method of claim 52, wherein the cartilage defect site is enzymatically treated prior to implantation of the scaffold or the periosteal or perichondrial tissue.

56. The method of claim 52 wherein the chondrocyte progenitor cells comprise mesenchymal stem cells.

57. A method for forming artificial cartilage, comprising:
delivering a tissue regenerative effective amount of light energy to an in vitro culture comprising a preparation of stromal cells and a substrate for attachment of cells; and
culturing the cells in a cell culture chamber for a time sufficient to produce artificial cartilage,
wherein delivering a tissue regenerative effective amount of light energy includes delivering light having a wavelength in the visible to near-infrared wavelength range and a power density of at least about 0.01 mW/cm² to the cells during culturing.

58. A method in accordance with claim 57, wherein the preparation of stromal cells comprises a combination of cells selected from the group consisting of chondrocytes, chondrocyte progenitor cells, fibroblasts, fibroblast-like cells, endothelial cells, pericytes, macrophages, monocytes, leukocytes, plasma cells, mast cells, adipocytes, umbilical cord cells, and bone marrow cells from umbilical cord blood.

59. The method of claim 58 wherein the chondrocyte progenitor cells comprise mesenchymal stem cells.

60. A method in accordance with claim 57, wherein the stromal cells are mammalian stem cells and the culturing further comprises culturing the stem cells for a time sufficient to allow them to differentiate into chondrocytes.

61. A method in accordance with claim 57, wherein the stromal cells are mammalian cells other than chondrocytes or chondrocyte stem cells and the culturing further comprises culturing the cells for a time sufficient to allow them to transdifferentiate into chondrocytes.

62. The method of claim 61, wherein the cells are fibroblasts and/or myocytes.

63. A method in accordance with claim 57, further comprising applying a shear flow stress between about 1 and about 100 dynes/cm² to the cells.

64. The method of claim 63, wherein the shear flow stress is between about 1 and about 50 dynes/cm².

65. The method according to claim 63, wherein the shear flow stress is applied by alternatingly creating a pressure differential across the substrate during culturing.

66. The method according to claim 63, wherein the shear flow stress results from applying a pressure differential to a fluid media across the substrate so that fluid media is forced through the substrate, wherein the fluid media comprises the growth medium.

67. A method in accordance with claim 57, wherein the cell culture chamber includes one or more light sources for delivering the tissue regenerative effective amount of light energy.
